(19) United States
(12) Patent Application Publication
(10) Pub. No.: US 2018/0200116 A1
(43) Pub. Date: Jul. 19, 2018

(54) PAD

(71) Applicants: Orin Tayeb, Gan Yavne (IL); Sivan Nomberg, Gan Yavne (IL)

(72) Inventors: Orin Tayeb, Gan Yavne (IL); Sivan Nomberg, Gan Yavne (IL)

(21) Appl. No.: 15/865,285

(22) Filed: Jan. 9, 2018

(30) Foreign Application Priority Data
Jan. 17, 2017 (IL) 250163

(51) Int. Cl. A61F 13/02; A61K 9/70

(52) U.S. Cl. CPC A61F 13/0206; A61K 9/7046; A61F 13/023; A61K 9/7092; A61F 13/0276

(57) ABSTRACT

In one aspect, the present invention is directed to a pad (10) comprising: a spongy layer (26), providing an activity; and a gluey and perforated layer (24), glued to the spongy layer (26), thereby allowing the spongy layer (26) to be in contact with a user's skin through the perforations of the gluey-perforated layer (24); wherein the area of said perforations in said gluey and perforated layer (24) is greater than the non-perforated area thereof; thereby providing a skin-attachable pad that provides the activity.

### TECHNICAL FIELD

The present invention relates to the field of pads, such as for medical use.

### BACKGROUND ART

Tinea cruris is an infection of the skin by Candida albicans. It can be found between folds in the human skin, such as the groin, scrotum, and the like, and be indistinguishable from fungal infections caused by tinia.

Tinea cruris is caused by continuous friction surfaces of skin, sweat secretion (which is an acidic substance), lack of ventilation, and more.

Tinea cruris symptoms include a rash, red, pink or brown, often accompanied by unbearable intense itching. This is a very common situation among men, and in particular, soldiers, athletes, diabetics and the obese.

On the market there exist inflammation-lowering medications, but they are absorbed in the skin and clothing of the patient, resulting in a situation wherein a few minutes after application, the medication (ointment, spray splashing etc.) is absorbed, the patient momentarily relieved, but remaining infected, without any solution.

It is an object of the present invention to provide a solution to the above-mentioned and other problems of the prior art.

Other objects and advantages of the invention will become apparent as the description proceeds.

### SUMMARY OF THE INVENTION

In one aspect, the present invention is directed to a pad (10) comprising:
a spongy layer (26), providing an activity; and
a gluey and perforated layer (24), glued to the spongy layer (26), thereby allowing the spongy layer (26) to be in contact with a user's skin through the perforations of the gluey and perforated layer (24);
wherein the area of said perforations in said gluey and perforated layer (24) is greater than the non-perforated area thereof;
thereby providing a skin-attachable pad that provides the activity.

Preferably, the pad further comprises an oilcloth layer (28), glued to the gluey and perforated layer (24), for retaining the gluey and perforated layer effective until use.

According to one embodiment of the invention, the gluey and perforated layer (24) is comprised of silicon.

Preferably, the gluey and perforated layer (24) is safe for human use.

Preferably, the perforations of the gluey and perforated layer (24) are between 60% to 99% of the area of the layer (24).

According to one embodiment of the invention, the contour of the pad is of a banana or crescent form.

According to one embodiment of the invention, the non-perforated area of the gluey and perforated layer (24) is in a net form.

According to one embodiment of the invention, the material of the gluey and perforated material is disposed on the margins.

According to one embodiment of the invention, the contour of the pad is rectangular, hexagonal or triangular, thereby allowing covering a larger area that the area of each pad, with no spaces therebetween.

According to one embodiment of the invention, the spongy layer (26) is made of 100% or other material that provides soft contact.

The spongy layer (26) may be made of opaque material, may comprises an evaporating material, sweat absorbing material, odor absorbing material, scent, hypoallergenic material, antiseptic material, inflammation-curable material, absorbed with evaporating material, and so on.

Dri-FIT is a material created by Nike enterprise for fitness apparel, consisting of a microfiber and polyester fabric engineered to evaporate sweat from the body. Nike enterprise makes socks, shirts, pants, shorts and many other accessories and apparel items out of Dri-FIT. No matter what clothing item it is used for, Dri-FIT moves sweat from the body to the surface of the fabric, where it can evaporate.

As such, the spongy layer (26) is preferably made of Dri-FIT fabric.

The pad may further comprise a smooth layer, for reducing friction with an opposite limb or the pad of an opposite limb. The layer may be of Teflon and the like.

In another aspect, the present invention is directed to a multiple-use pad (40), comprising:
a reusable pad comprising:
   a one-time assembly (42) having a spongy layer comprising first and second perforated and gluey layers (24), on each side thereof;
   a reusable assembly (44) having a friction-relievable layer to which a third perforated and gluey layer (24) is attached;
   wherein the perforated and gluey layers correspond and overlap to each other by the form and layout thereof;
thereby when attaching the one-time assembly (42) to the reusable assembly (44), a gluey layer of the one-time assembly touches the gluey layer of the reusable assembly and the perforations of the assemblies overlap each other.

The fact that the gluey layers touch each other (in contrast to touching a fabric) prolongs the lifetime of the reusable assembly. The fact that the perforations of the one-time and reusable assemblies overlap each other leaves the perforations unsealed.

The reference numbers have been used to point out elements in the embodiments described and illustrated herein, in order to facilitate the understanding of the invention. They are meant to be merely illustrative, and not limiting. Also, the foregoing embodiments of the invention have been described and illustrated in conjunction with systems and methods thereof, which are meant to be merely illustrative, and not limiting.

### BRIEF DESCRIPTION OF DRAWINGS

Preferred embodiments, features, aspects and advantages of the present invention are described herein in conjunction with the following drawings:

FIG. 1 is a side view of an individual while walking, wherein two arrows point the areas in which Tinea cruris symptoms may develop.

FIG. 2 is a perspective view schematically illustrating a pad 10, according to one embodiment of the invention.

FIG. 3 further details the structure of pad 10 of FIG. 2.

FIG. 4 is a top view schematically illustrating a pad 20, according to another embodiment of the invention.

FIG. 5 is a top view schematically illustrating a pad 30, according to yet another embodiment of the invention.

FIG. 6 is a top view of a pad 50 generated from smaller pads 30, according to one embodiment of the invention.

FIG. 7 schematically illustrates a roller 16 which is constructed from the layers of a pad, according to one embodiment of the invention.

Each of FIGS. 8a and 8b is a sectional view schematically illustrating the layers of the pad 10 of FIG. 2.

FIGS. 9a to 9c illustrate steps in using pad 10 of FIG. 2. FIG. 9a is a sectional view illustrating the pad of FIG. 2.

Each of FIGS. 10 and 11 is a cross-section illustrating the structure and usage of a multiple-use pad 40, according to one embodiment of the invention.

It should be understood that the drawings are not necessarily drawn to scale.

### DESCRIPTION OF EMBODIMENTS

The present invention will be understood from the following detailed description of preferred embodiments (“best mode”), which are meant to be descriptive and not limiting. For the sake of brevity, some well-known features, methods, systems, procedures, components, circuits, and so on, are not described in detail.

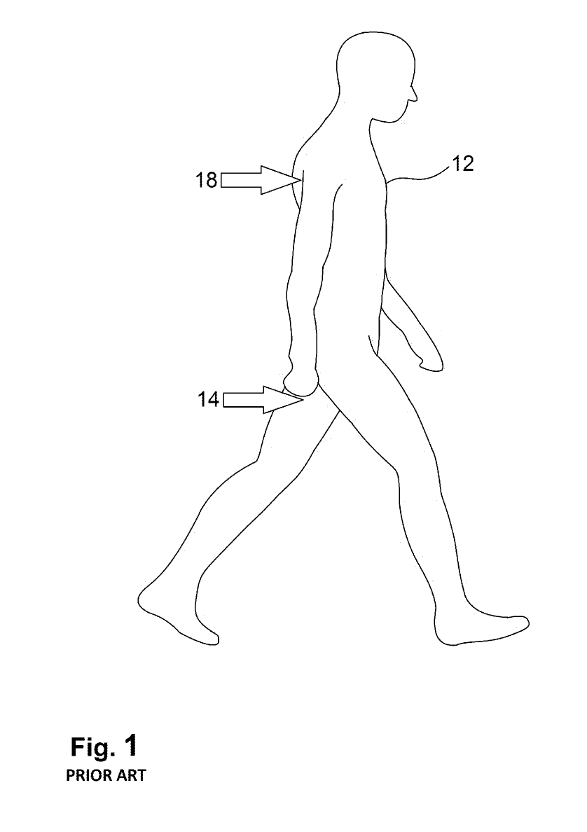

FIG. 1 is a side view of an individual while walking, wherein two arrows point to areas in which Tinea cruris symptoms may develop.

Arrow 14 points to the groin area, and arrow 18 points to the armpit area of the individual. Arrow 14 shows the groin and inner thighs. Arrow 18 shows the armpit area. These areas often suffer from effects of friction because two organs rub against each other. Also, in these friction-prone regions, sweat does not evaporate, resulting in inflammation.

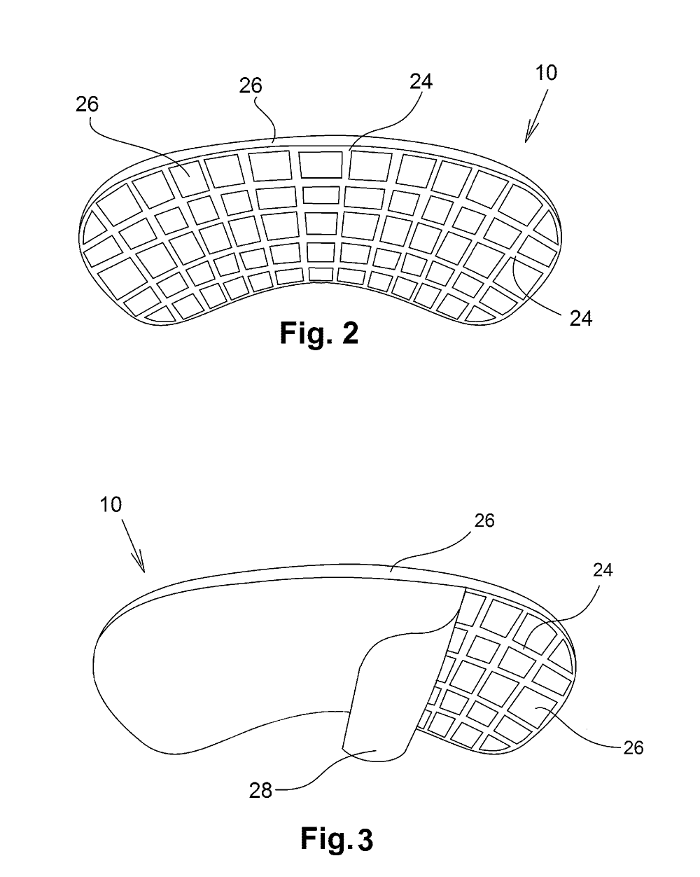

FIG. 2 is a perspective view schematically illustrating a pad 10, according to one embodiment of the invention.

The shown side is the side to be glued to the skin of a user. In the figure is shown the spongy layer 26 which absorbs sweat. The spongy layer 26 may also be absorbed with an active substance such as disinfectant, inflammation relief substance, and so on.

On the spongy layer 26 is glued a gluey pattern 24, for gluing the pad 10 to the skin of a user. Preferably, the gluey pattern 24 is in a form of a net, and made of silicon glue.

The gluey pattern has “holes” through which the spongy layer 24 comes into contact with the skin of a user. This structure allows two operations in one pad: the gluey pattern 24 allows gluing the pad to a user's skin, while the spongy layer 26 is the active substance, i.e., can absorb sweat, can comprise sweat evaporating substance, and the like.

FIG. 3 further details the structure of pad 10 of FIG. 2.

In the figure is shown an oilcloth 28 attached to the glue net 24 from the other side thereof. As a result of the nature of an oilcloth, it can be separated “easily” from the glue net 24. The oilcloth layer 28 keeps the gluey pattern 24 effective until use.

The length of the pad is between 8 cm to 12 cm, depending on the size of a user thereof. Thus, a user may select the desired size from a plurality of available sizes.

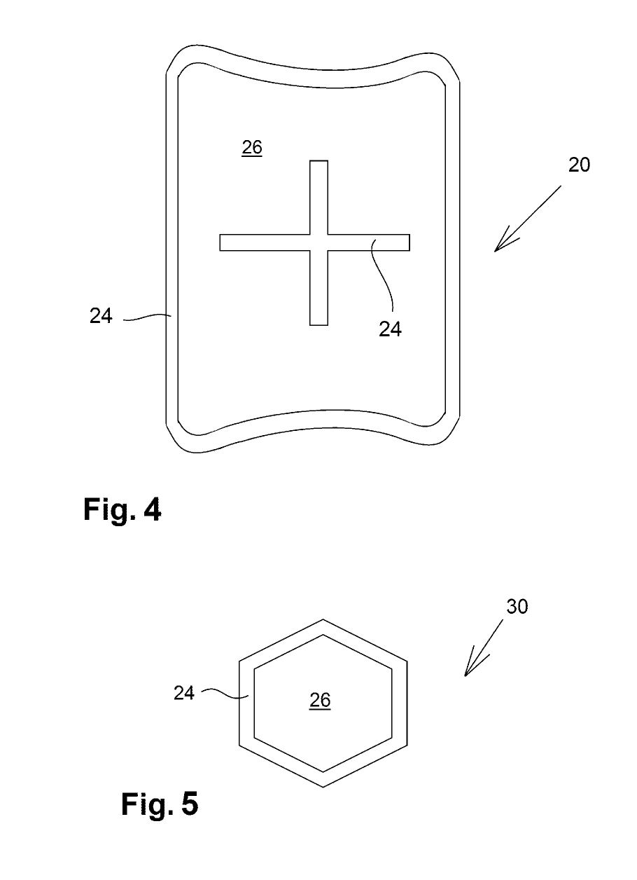

FIG. 4 is a top view schematically illustrating a pad 20, according to another embodiment of the invention.

The contour of the pad corresponds to the contour of a human groin.

As per the structure, as illustrated, the gluey pattern 24 comprises the margins of the pad 20. In addition, the pattern comprises a cross, for attaching the center of the pad to a human body.

FIG. 5 is a top view schematically illustrating a pad 30, according to yet another embodiment of the invention.

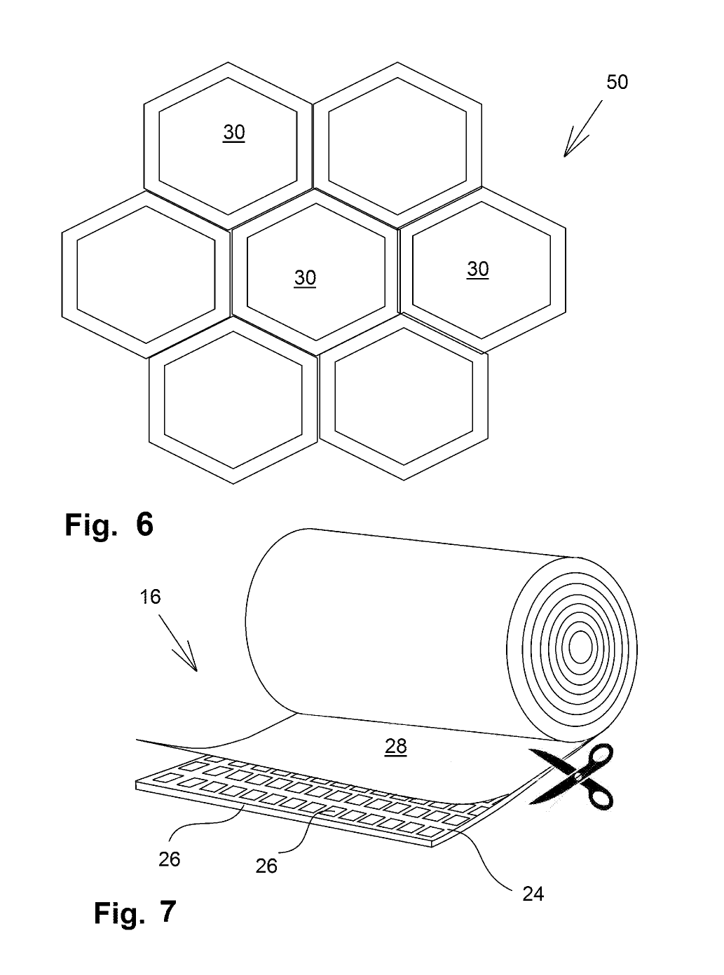

According to this embodiment of the invention, the contour of pad 30 is in a hexagonal form. The size of the pads is “small” (e.g., 2 to 4 cm), thereby allowing generating a pad of a desired size by attaching a plurality of pads 30 adjacently to each other, as illustrated in FIG. 6.

FIG. 7 schematically illustrates a roller 16 which is constructed from the layers of a pad, according to one embodiment of the invention.

The roller comprises the layers mentioned above: a spongy layer 26 (the active layer), a gluey pattern 24 glued to the active layer 26, and an oilcloth layer 28. A user may cut from the roller a desired size of pad.

Each of FIGS. 8a and 8b is a sectional view of the pad 10 of FIG. 2. They show the three layers: The gluey and perforated layer 24, to which the spongy layer 26 is glued from one side thereof, and the oilcloth layer 28 from the other side.

The difference between FIGS. 8a and 8b is that in FIG. 8b the gluey and perforated layer 28 is slightly immersed in the spongy layer 26. The immersion may be carried out in the production process by slightly heating the gluey and perforated layer when attached to the spongy layer.

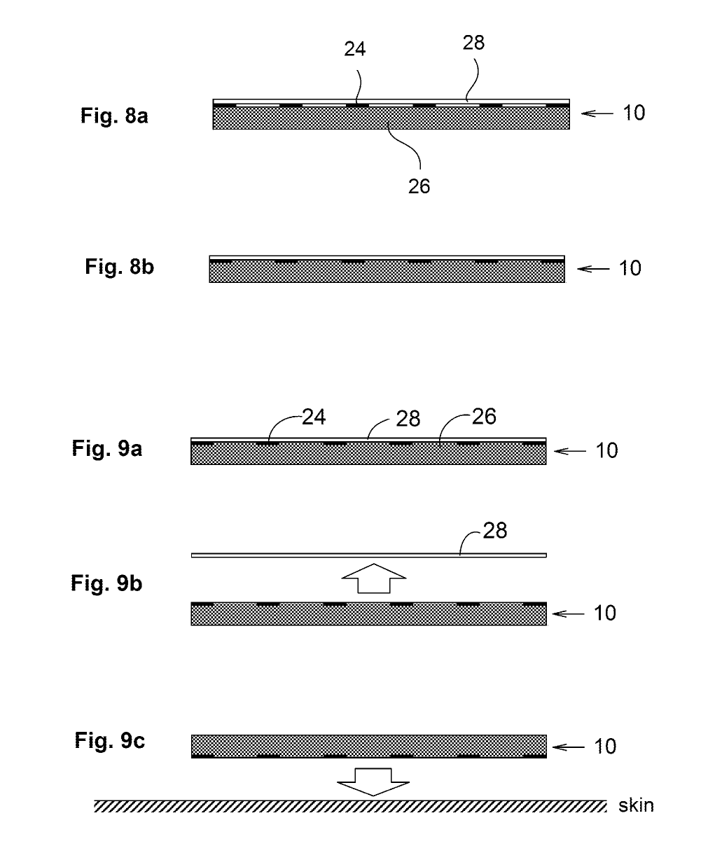

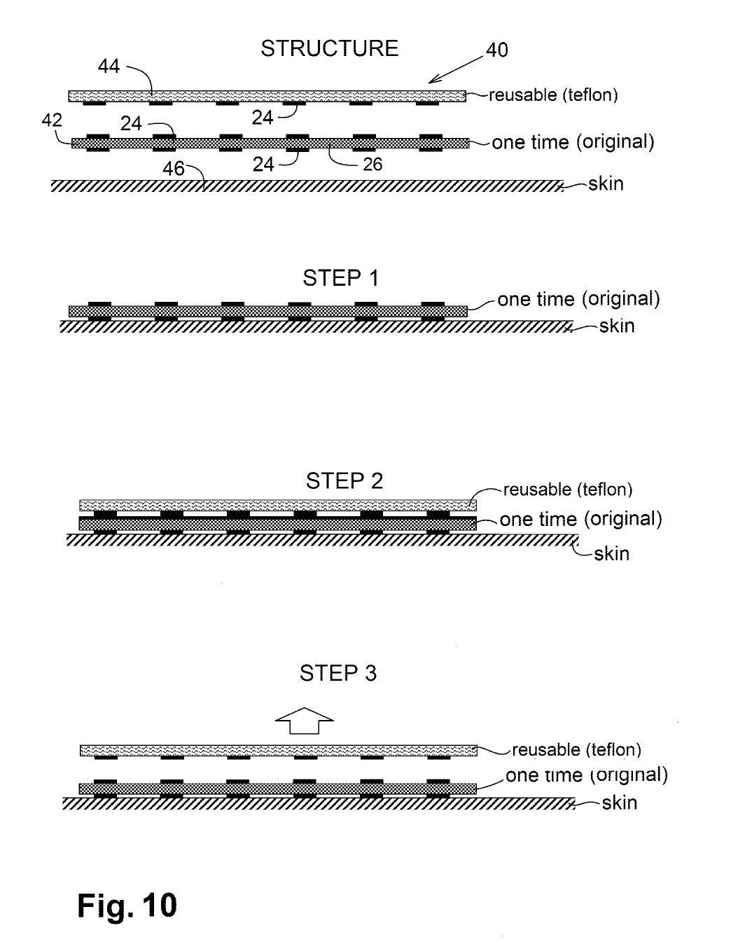

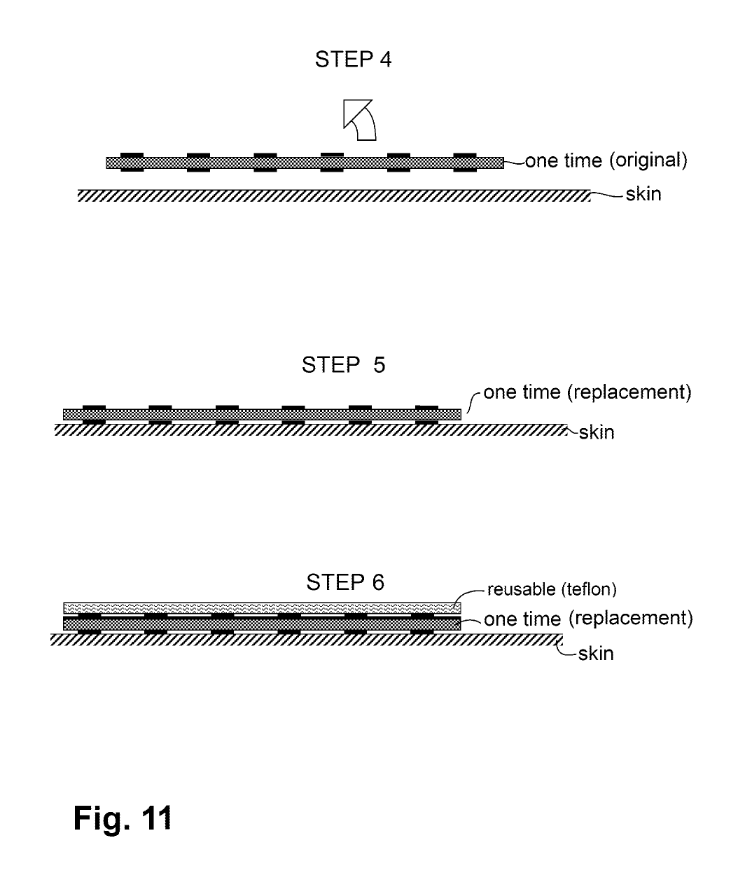

FIGS. 9a to 9c illustrate steps in using pad 10 of FIG. 2. FIG. 9a is a sectional view illustrating the pad of FIG. 2.

In FIG. 9b, a user removes the oilcloth layer 28. In FIG. 9c the user turns the pad 10 upside down, and attaches it to the skin of an organ. Since the gluey and perforated layer 24 touches the skin, the pad sticks to the skin.

Each of FIGS. 10 and 11 is a cross-section illustrating the structure and usage of a multiple-use pad 40, according to another embodiment of the invention.

As per the structure, the multiple-use pad 40 comprises two assemblies: a one-time assembly 42 and a reusable assembly 44.

The one-time assembly 42 is the assembly that comes into contact with the skin 46 of a user organ, and as such it must often be replaced, i.e., it is oriented for a one-time use. The one-time assembly 42 comprises two parallel gluey and perforated layers 24, one of each side thereof.

The object reusable assembly 44 is to provide pleasant contact with the opposite organ (reducing friction, soft contact, and the like), whether it is bare, or in the case wherein another pad is attached thereto. Such a layer may comprise 100% cotton, Teflon, and the like.

Each of the assemblies 42 and 44 may comprise an oilcloth (not illustrated) for retaining the affectivity of its gluey and perforated layer 24 until use.

At STEP 1 a one-time assembly is attached to the skin of a user organ. This one-time assembly is referred to herein as “original” since it will be replaced later with another one-time assembly of the same kind, which will be referred to as “replacement”.

At STEP 2, a reusable assembly is attached to the one-time assembly. The user leaves the pad in this situation, until he decides to replace the original one-time assembly with a replacement one-time assembly

In order to replace the original one-time assembly, at STEP 3, the reusable assembly is detached from the original one-time assembly.

At STEP 4 the original one-time assembly is disposed of.

At STEP 5 a replacement one-time assembly is attached to the skin of a human organ.

And at STEP 6 the multiple times assembly is attached to the replacement one-time assembly.

This process can be carried out for a few “sessions”, until the glues of the multiple times assembly loses its effectiveness.

It should be noted that when the one-time assembly is attached to the reusable assembly, the gluey pattern of one assembly is attached to the gluey pattern of the other assembly. As such, assuming the gluey patterns are made of silicon glue, one silicon pattern touches only the other silicon pattern, thereby prolonging the stickiness of the gluey layers.

In the figures and/or description herein, the following reference numerals (Reference Signs List) have been mentioned:
numeral 10 denotes a one-time pad, according to one embodiment of the invention;
numeral 12 denotes an individual;
numeral 14 denotes a groin of an individual;
numeral 18 denotes an armpit area of an individual;
numeral 20 denotes a one-time pad, according to another embodiment of the invention;
numeral 24 denotes a gluey pattern;
numeral 26 denotes a spongy layer 26 of a pad, which absorbs sweat and actually is an “active layer”;
numeral 28 is an oilcloth layer;
numeral 30 denotes a one-time pad, according to yet another embodiment of the invention;
numeral 40 denotes a multiple-use pad, according to one another embodiment of the invention;
numeral 42 denotes a one-time assembly, of a multiple-use pad 40;
numeral 44 denotes reusable assembly, of a multiple-use pad 40; and
numeral 50 denotes a pad generated from smaller pads.

The foregoing description and illustrations of the embodiments of the invention has been presented for the purposes of illustration. It is not intended to be exhaustive or to limit the invention to the above description in any form.

Any term that has been defined above and used in the claims, should to be interpreted according to this definition.

The reference numbers in the claims are not a assembly of the claims, but rather used for facilitating the reading thereof. These reference numbers should not be interpreted as limiting the claims in any form.

1. A pad (10) comprising:
a spongy layer (26), providing an activity; and
a gluey and perforated layer (24), glued to said spongy layer (26), for allowing said spongy layer (26) to be in contact with a user's skin through the perforations of said gluey and perforated layer (24);
wherein the area of said perforations in said gluey and perforated layer (24) is greater than the non-perforated area thereof;
thereby providing a skin-attachable pad that provides said activity.

2. A pad according to claim 1, further comprising an oilcloth layer (28), glued to said gluey and perforated layer (24), for retaining said gluey and perforated layer effective until use.

3. A pad according to claim 1, wherein said gluey and perforated layer (24) comprises silicon.

4. A pad according to claim 1, wherein the perforations of said gluey and perforated layer (24) are between 60% to 99% of the area of said gluey and perforated layer (24).

5. A pad according to claim 1, wherein a contour thereof is of a banana crescent form.

6. A pad according to claim 1, wherein the non-perforated area of said gluey and perforated layer (24) is in a net form.

7. A pad according to claim 1, wherein the material of said gluey and perforated material is disposed only on the margins.

8. A pad according to claim 1, wherein the contour of said pad is rectangular, or hexagonal or triangular, thereby allowing covering a larger area that the area of each pad, with no spaces therebetween.

9. A pad according to claim 1, wherein said spongy layer (26) is made of 100% cotton or other material that provides soft contact.

10. A pad according to claim 1, wherein said spongy layer (26) is made of light opaque material.

11. A pad according to claim 1, wherein said spongy layer (26) comprises an evaporating material.

12. A pad according to claim 1, wherein said spongy layer (26) comprises sweat-absorbing material.

13. A pad according to claim 1, wherein said spongy layer (26) comprises odor-absorbing material.

14. A pad according to claim 1, wherein said spongy layer (26) comprises hypoallergenic material.

15. A pad according to claim 1, wherein said spongy layer (26) comprises antiseptic material.

16. A pad according to claim 1, wherein said spongy layer (26) comprises a scent material.

17. A pad according to claim 1, wherein said spongy layer (26) comprises inflammation-curable material.

18. A pad according to claim 1, wherein said spongy layer (26) is made of a microfiber and polyester fabric engineered to evaporate sweat from the body.

19. A pad according to claim 1, wherein said spongy layer is absorbed with evaporating material.

20. A pad according to claim 1, further comprising a smoother layer that the other layers of said pad, for reducing friction with an opposite limb or a pad of an opposite limb.

21. A multiple-use pad (40), comprising:
a reusable pad comprising:
a one-time assembly (42) having a spongy layer comprising a first and second perforated gluey layers (24), on each side thereof;
a reusable assembly (44) having friction-relievable layer to which a third perforated gluey layer (24) is attached;
wherein the perforated gluey layers correspond and overlap to each other by the form and layout thereof;
thereby when attaching said one-time assembly (42) to said reusable assembly (44), a gluey layer of said one-time assembly touches the gluey layer of said reusable assembly and the perforations of the assemblies overlap each other.
